(19) United States
(12) Patent Application Publication
(10) Pub. No.: US 2020/0367864 A1
(43) Pub. Date: Nov. 26, 2020

(54) INTEGRATED URINE TESTING DEVICE

(71) Applicant: Deep Bajaj, Guragon (IN)

(72) Inventor: Deep Bajaj, Guragon (IN)

(21) Appl. No.: 16/966,308

(22) PCT Filed: Jan. 23, 2019
(86) PCT No.: PCT/IN2019/050050

§ 371 Date: Jul. 30, 2020

(30) Foreign Application Priority Data
Jan. 30, 2018 (IN) 201811003457

(51) Int. Cl. A61B 10/00

(52) U.S. Cl. CPC A61B 10/007; A61B 2560/04

(57) ABSTRACT

The present disclosure relates to an integrated urine testing device (100) to facilitate the female user to urinate in the standing or sitting or squatting positions and allow them to determine the analyte present in the urine for testing purpose, for example, pregnancy. The device (100) includes the foldable structure (102), defining a spout member (106) having the inlet opening (108) and the outlet opening (112). The device (100) includes slits (126) and the test strip (132). The test strip (132) is fixedly disposed at the slits (126) of the wall member (118) of the spout member (106). The test strip (132) include the testing portion (134) is adapted to come in contact with the urine flowing in the spout member (106), and the indicating portion (136) of the test strip (132) is adapted for indicating test results without the test strip (132) being removed from the device (100).

### FIELD OF THE INVENTION

The present disclosure relates to a diagnostic device. More particularly relates to an integrated urine testing device to facilitate a female user to urinate in a standing position or a sitting position or a squatting position and allow them to determine an analyte present in the urine for testing purpose, for example, pregnancy.

### BACKGROUND OF THE INVENTION

In healthcare and medical fields, widely used diagnostic methodology for determining health condition and other disorders or diseases is to test body fluids majorly including, but not limited to, blood, saliva or sputum and urine. For example, the urine sample may include number of analyte that may be used as an indicators of present health condition of a person from whom the sample being received. Common urine based tests typically include screening for and/or diagnosis of heath conditions including, but not limited to, Urinary Tract Infections (UTI), kidney disorders, liver ailments, diabetes, metabolic condition, urine pH, protein, blood cells, hormones, pregnancy etc.

The urine based testing method will typically include collecting the urine into a sample collector (for example: a plastic bottle or a container) and thereafter taking the container to a laboratory for analysis, or for home based tests, using a dropper for analysis in a cassette based device, or dipping a test strip in the container in a precise manner to a fixed point beyond which it gets soiled and test results may be inaccurate. The process of urine collection by filling the container by directing the urine into the container may found to be uncomfortable and awkward actions to perform. Dipping the test strip and waiting for results may also found to be uncomfortable and awkward actions to perform. Some of the tests may even require holding the test strip in the direction of urine flow while urinating and removing the test strip to see the result. While performing these actions, the user may spoil the test strip or the result provided by the test strip may be inaccurate since the test strip may not be accurately positioned with respect to the urine flow and the user may also soil their hands posing hygiene issues during testing procedures.

Another common problem faced during the urine collection and testing by the users include, but not limited to, hygiene of collection chambers or rooms like, toilets. This is because, the urine collection is typically carried-out in a seated position or a squat position on a toilet seat (western or modern commodes) and the toilet seats are not often cleaned due to many reasons like, number of users, and continuous usage. Also, the users are reluctant to sit or use the unhygienic toilets since there may a risk of getting infected with diseases. However, in order to avoid the contact of unhygienic toilet seats by sitting on them, the users (for example: female individual) may position themselves in a squatting position to collect the urine sample. Thus, the urine collection methods presently being carried-out are awkward, difficult and do not allow targeted urine collection and can easily result in soiling of hands, clothes and the toilet area. Also, such collection methods are not suitable for disabled or aged people or people having a disease such as arthritis. Thus, there is a need for a urinary device which allows the users, particularly female users to urinate in a standing position or a sitting position, as per their convenience and can simultaneously allow them to do the urine analysis without the use of any extensions or without requiring to remove any part of the device for example the test strip for knowing the test result.

One kind of existing device for testing of pregnancy used in home is based on a plastic cassette, where a test strip is used with the plastic cassette. In the plastic cassette based testing, process is to sit on a pot, fill the urine in a tumbler/plastic bottle, using a dropper to get urine and then putting drops on pregnancy cassettes. Further, in paper strip based testing, the process is to sit on a pot, fill the urine in a tumbler/plastic bottle, then inserting the strip in bottle till the restricted or highlighted area. This may often soil for 5 to 10 seconds and requires a waiting time for about 5 minutes and may also require a specific arrangement for obtaining an accurate result. The process however is found to be unhygienic in nature. Therefore, the existing testing methods are inconvenient, clumsy, regressive and most of all unhygienic.

One kind of device for assisting female urinating is disclosed in U.S. Pat. No. 3,329,973 (hereinafter referred to as patent '973). The patent '973 relates to a single-use throw-away urinal of inexpensive construction adapted to be cast into a toilet bowl and flushed away owing to its loss of strength when its exterior comes in contact with water. However, the device is used an alternative for urinating when the user found that the toilets are not clean and hygienic for use.

Another kind of device for assisting female urination is disclosed in U.S. Pat. No. 4,608,046 (hereinafter referred to as the patent '046). The device in the patent '046 is a urinary aid for use by females. The device includes a flat-folded tube having an end insertable between the labia. The tube is expandable by light hand squeezing to open the inserted end of the tube to a urine-receptive configuration which spread the labia and in which the device may be pressed against the urinary meatus. However, the device in the patent '046 only facilitates urination by females in circumstances where toilet facilities are unavailable or, if available are unsanitary or otherwise undesirable.

Yet another kind of device for assisting female urination is disclosed in U.S. Pat. No. 5,742,948 (hereinafter referred to as the patent '948) and U.S. Pat. No. 5,408,703 (hereafter referred to as the patent '703) relates to a disposable female urination devices having a main body portion with two openings. One opening is adapted to fit over a female urethral area, while the other opening is used to discharge urine. However, the devices in the patent '948 and the patent '703 again are assisting urination of females when they find that the toilets are not clean and hygienic for use.

Yet another device for assisting female urination and urine analysis is disclosed in U.S. Pat. No. 5,605,161A (hereinafter referred to as the patent '161). The device disclosed in the patent '161 becomes pliant after contacting with water for a short time and thus there is possibility of leakage or spillage of urine during its usage itself. Also, the user may require special accommodations for holding the device in a proper position during urination against the body. Also, loops provided in device of the patent '161 are glued adjacent to a rim and thus during testing procedure the loops may be detached from the rim resulting in difficulty to gather the urine sample.

Yet another device for assisting female urination and urine analysis is disclosed in DE4411824C2 (hereinafter referred to as the patent '824). The device in the patent '824 consist of a flexible disposable material. The disadvantage of this device is that, it requires an additional steering device for focusing the beam flow. Also, the additional steering device in the patent '824 is placed in a longitudinal direction and thus the urine affects the strip.

Yet another device for assisting female urination and urine analysis is disclosed in WO2015175301 (hereinafter referred to as the patent '301). The device is a flexible, integrated, urine-based diagnostic device designed such that one or more diagnostic channels are integrated into the body of the device. The flexibility in the device of the patent '301 may cause deformation of the device during storing, packaging and even during testing procedure. Thus the device in the patent '301 is unsanitary and inconvenient to use.

Yet another device for assisting female urination and urine analysis is disclosed in U.S. Pat. No. 6,403,298 (hereinafter referred to as the patent '298). The urine self-testing device in the patent '298 is placed directly into the toilet after urination which avoids the direct placement and retention of an apparatus in the stream of urine. As the device in the patent '298 is placed directly on the toilet, the device may be exposed to the germs and bacteria present on the toilet and the results obtained may not be accurate.

Yet another device for assisting female urination and urine analysis is disclosed in GB2415386 (hereinafter referred to as the patent '386). The device in the patent '386 includes a urine testing element which is releasably attached to the device. The testing element in the device of the patent '386 after passage of urine by the user needs to be removed in order to see and obtain the test results. Thus, the hands or clothes of the user gets soiled. Also, as the testing element is positioned at an inlet of the device, the testing element itself may become a hindrance or may even fall-off into the device due to contact of the body portion of the user to the testing element. Further, means for releasably attaching the device may not hold the testing element firmly during packaging, and usage since the device includes only one means for releasably attaching the testing element. Thus the testing element in the device of the patent '386 may be difficult to retrieve if it is released or fallen into the device. In addition, a user for using the device in the patent '386 has to buy and assemble the test strip separately. Many a times, the user may not find the desired test strip due to non-availability of desired test strip and the user may feel cheated. Also, since the test strip is independently bought and assembled, the process of assembling becomes a tedious job for the user and may find dangerous as the test strip and the device might get damaged while inserting or installing the test strip into the device and also while removing the test strip from the device for seeing the test result. The open end of the device in the patent '386 is more prone to spillage or leakages since the open end does not conform to the exterior surface of the urinary body part and may cause injuries as well.

Many attempts have been made in the past to provide urine collecting or directing device for urine analysis, but these devices are either difficult to use or need assembly by the user, are awkward and unsanitary or even have rudimentary method for testing and using the device. Some of these devices need a particular setting for construction which adds up to the cost of the device. Also, the commonly used testing methods require use of plastic based materials or non-biodegradable materials which will end up in landfills and cause plastic pollution.

The present disclosure is directed to overcome one or more of the problems as set forth above.

### OBJECTIVES OF THE INVENTION

The main objective of the present disclosure is to provide an integrated urine testing device which facilitate urine analysis without spillage of urine on user's body or clothes while using by the user in a standing position or seated/sitting position or squatting position and without using any other additional accessory.

Another objective is to provide the urine testing device having simplified process with a locking mechanism. The locking mechanism is a small strip like Aluminum or Copper or Brass or Stainless steel which can be inserted/affixed during the manufacturing process right at the top surface of device to ensure the grip and shape stays firm.

Still another objective is to provide a device that can be easily used in standing or sitting or squatting positions whichever is convenient for the user.

Yet another objective is to provide a urine testing device which is easy to use due to its symmetrical structure without requiring a high accuracy of alignment with the urethral area of a female user.

### SUMMARY OF THE INVENTION

The said objectives are achieved by the urine testing device as proposed in the present disclosure. The urine testing device is for a female user. The urine testing device includes a foldable structure being formed from a sheet of material, defining a spout member having an inlet opening at a proximal end and an outlet opening at a distal end. The spout member includes a top member and wall members. The inlet opening is being adapted to be positioned abutting an exterior surface of a female urinary part to receive urine at the inlet opening and to discharge the urine through the outlet opening. The urine testing device further includes at least two slits configured on any one of the wall members of the spout member. The slits are being configured proximal to the inlet opening of the foldable structure. The urine testing device further includes at least one test strip having a testing portion and an indicating portion. The test strip is being fixedly disposed at the slits of the wall member of the spout member. The testing portion of the test strip is disposed inside the spout member and is adapted to come in contact with the urine flowing in the spout member for testing the urine, and the indicating portion of the test strip is disposed outside the spout member for indicating the test results without the test strip being removed from the testing device.

In an embodiment, the inlet opening has a larger size than the outlet opening forming a frustum of pyramid structure.

In an embodiment, the inlet opening is provided with an absorbent material around its periphery for absorbing any possible spilled urine during urination.

In an embodiment, the inlet opening has a profile adapted to conform to a body portion of the exterior surface of the female urinary part during urination and thereby creating an effective seal to prevent splashing or spillage of the urine during urination in a standing position or sitting position or squatting position.

In an embodiment, the foldable structure has one or more longitudinal creases for folding the urine testing device before usage and for forming the wall members of the foldable structure of the testing device during the usage.

In an embodiment, the wall members of the foldable structure have at least one locking mechanism on its surface, and wherein the locking mechanism is adapted to prevent folding of the urine testing device during the usage, wherein the locking mechanism is a metallic strip which is inserted or affixed during manufacturing process right at top surface or outer surface of the device to ensure the grip and shape of the device stays firm.

In an embodiment, the slits in the wall member are configured in such a way that the test strip being disposed in the slits are positioned substantially vertically with respect to an axis of the spout member of the urine testing device such that the testing portion of the test strip is substantially perpendicular to flow path of the urine.

In an embodiment, the slits in the wall member are configured in such a way that the test strip being disposed in the slits are positioned substantially parallel with respect to the axis of the spout member of the urine testing device such that the test strip is substantially parallel to flow path of the urine and the urine is allowed to flow over the testing portion of the test strip.

In an embodiment, the slits are provided with a hydrophobic layer for preventing spillage of urine through the slits.

In an embodiment, the slits are provided with relieving holes on either ends of the slits for providing tear resistance during insertion of the test strip into the slits.

In an embodiment, the material of the foldable structure is a biodegradable material, like cardboard.

In an embodiment, the spout member has an inner surface coated with a hydrophobic material.

In an embodiment, the outlet opening is provided with an elongated channel having a v-shaped cross section for guiding and increasing flow rate of the urine.

### BRIEF DESCRIPTION OF THE ACCOMPANYING DRAWINGS

FIG. 1 shows a front perspective view of a urine testing device formed by a foldable structure, according to an exemplary embodiment of the present disclosure;

FIG. 2 shows a rear perspective view of the urine testing device shown in FIG. 1, according to an exemplary embodiment of the present disclosure;

FIG. 3 shows a side view of the urine testing device shown in FIGS. 1 and 2, according to an exemplary embodiment of the present disclosure;

FIG. 4 shows a top view of the urine testing device shown in FIGS. 1 to 3, according to an exemplary embodiment of the present disclosure;

FIG. 5 shows a rear view of the urine testing device shown in FIGS. 1 to 4, according to an exemplary embodiment of the present disclosure;

FIG. 6 shows a side view of the urine testing device shown in FIGS. 1 to 5, to show the device being brought to workable form, according to an exemplary embodiment of the present disclosure;

FIG. 7A shows a schematic diagram of magnified portion “W” of a wall member of the device shown in FIG. 2, according to an exemplary embodiment of the present disclosure;

FIG. 7B shows a schematic diagram of another embodiment of the magnified portion “W” of the wall member of the device, according to an exemplary embodiment of the present disclosure; and

FIGS. 8A-8E show various views of a urine testing device, according to another exemplary embodiment of the present disclosure.

### DETAILED DESCRIPTION OF THE DISCLOSURE WITH REFERENCE TO ACCOMPANYING DRAWINGS

Provided below is a non-limiting exemplary embodiment of the present invention and a reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claim.

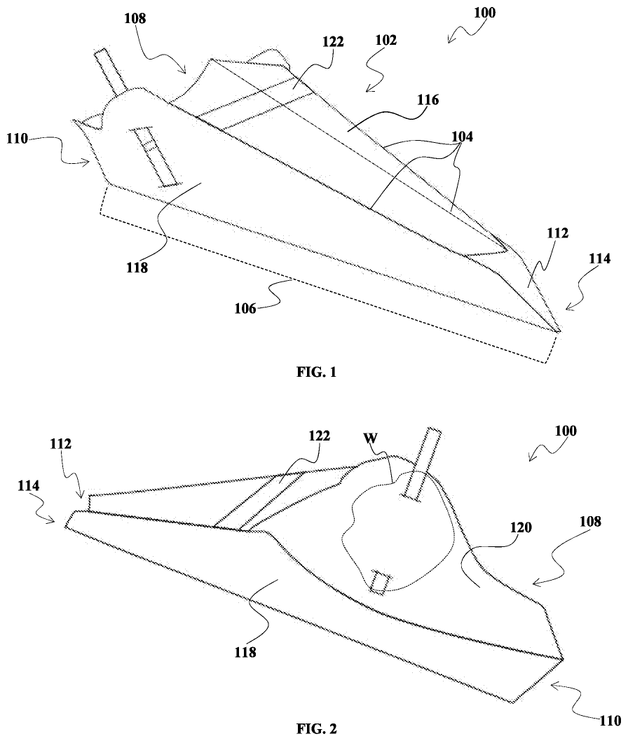

FIGS. 1 and 2 illustrate a front perspective view and a rear perspective view of a urine testing device (100), according to an exemplary embodiment of the present disclosure. Terms “urine testing device”, “testing device” and “device” are interchangeably used in the present disclosure. It should be understood that, the terms “urine testing device”, “testing device” and “device” are same and they are relating to a same device. The testing device (100) as disclosed in the present disclosure can be used by users, including, but not limited to, a person having arthritis, aged or older persons, a female individual, and a female athlete. The testing device (100) can be used by the user in positions such as standing position (not shown), a sitting or seated position (not shown), squatting position (not shown). The position for usage of the device (100) may be dependent on various factors like, physical strength and hygiene condition of a toilet (not shown).

The testing device (100) as illustrated in the FIGS. 1 and 2 includes a foldable structure (102) formed from a sheet of material. The material of the foldable structure (102) is a biodegradable material for example, paper, paper board or card board, biodegradable plastic. Thus, the device (100) is an eco-friendly device. The foldable structure (102) is provided with one or more longitudinal creases (104) by way of phantom lines in the sheet of material before the sheet of material being assembled to form the foldable structure (102). In one embodiment, the longitudinal creases (104) of the foldable structure (102) can be folded at a manufacturing facility by a machine before the usage and during packaging. Thus forming a flattened or compacted structure or in a collapsed configuration.

In the illustrated embodiment, after taking out the compacted structure of the device (100) from the packaging the longitudinal creases (104) are folded to form the testing device (100) for conducting a test. That is to say, when the testing device (100) is in non-deployed or non-working state, the testing device (100) can be stored in a flattened manner or compacted manner (not shown) with the help of the longitudinal creases (104). And when the device (100) is required to be brought to the working or open configuration from the flattened or compacted manner, a little pressure can be applied by the user on the longitudinal creases (104) to push the longitudinal creases (104) towards one another to make the device (100) to a workable form. The foldable structure (102) thus formed is converted into a spout member (106) of the device (100).

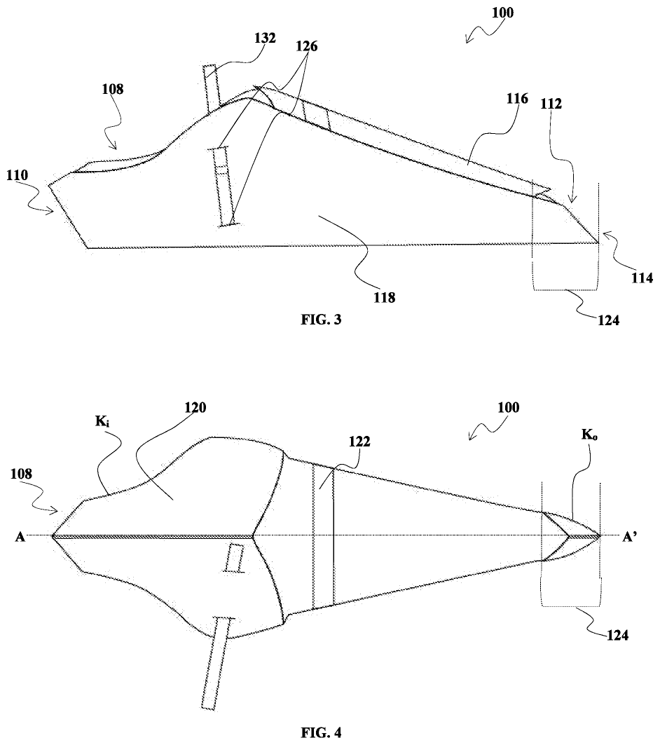

FIGS. 3 and 4 illustrate a side view and a top view of the urine testing device (100) illustrated in FIGS. 1 and 2 respectively, according to the exemplary embodiment of the present disclosure. The spout member (106) defined by the foldable structure (102) includes an inlet opening (108) at a proximal end (110) of the device (100) and an outlet opening (112) at a distal end (114) of the device (100). The proximal end (110) of the device (100) as referred in the present disclosure should be understood that the end of the device (100) which is closer to the user or an exterior surface of a urinary body part of the user. Similarly, the distal end (114) of the device (100) as referred in the present disclosure should be understood as an opposite end of the proximal end (110) and it is located farther or away from the proximal end (110).

The spout member (106) includes a top member (116) and wall members (118) between the inlet opening (108) and the outlet opening (112). In the illustrated embodiment shown in FIGS. 1 to 5, the device (100) includes two wall members (118) and one top member (116) formed due to pressing of the longitudinal creases (104) of the foldable structure (102). The device further includes an inner surface (120) (more clearly shown in FIG. 2) formed by the wall members (118) and the top member (116). It may be contemplated that, the device (100) may be configured to have more than two wall members (118) and a base member (not shown) opposite to the top member (116). In an embodiment, the inner surface (120) of the spout member (106) can be coated with a hydrophobic material, where the hydrophobic coating does not allow the urine to wet the device (100) and thus allows the device (100) to retain the its shape even after passage of urine into the device (100).

The wall members (118) of the foldable structure (102) further includes at least one locking mechanism (122) on its outer surface of the top member (116) to prevent folding of the testing device (100) when it is brought in to a deployed or workable form (shown in FIG. 6) and during the usage of the device (100). The locking mechanism (122) is a small strip for example, a metallic strip which can be inserted or affixed during the manufacturing process right at top surface or outer surface of the device (100) to ensure the grip and shape of the device (100) stays firm. In an exemplary embodiment, the strip can be made of material including, but not limited to, metals or alloys made of Aluminum, Copper, Brass, Stainless Steel. The stiffness of the strip causes the device to be maintained in the deployed or workable form till the user completes the usage of the device (100). In another embodiment, the strip can be made of any other non-metallic material or any other material which can provide stiffness to the top member (116) when the device is deployed or brought to the workable form. The strip may be affixed on the top member (116) by an adhesive. In the illustrated embodiment, the device (100) is configured in to a frustum of pyramid structure, where the inlet opening (108) is having larger size than the outlet opening (112) and thus the flow of urine in the device (100) happens smoothly and due to gravity. That is to say, an area (Kᵢ) of inlet opening (108) is larger than an area (Kₒ) of the outlet opening (112) (shown in FIG. 4). Further, the inlet opening (108) can be provided with an absorbent material (109) around its periphery for absorbing any possible spilled urine during urination and testing procedure. The absorbent material (109) may be a cotton material or a cellulosic material which are biodegradable.

In the illustrated embodiment, the outlet opening (112) is provided with an elongated channel (124) projecting outwardly from the user's body and clothes. The elongated channel (124) has a v-shaped cross section for guiding, increasing flow rate of the urine and to direct the urine flow to the farthest extent possible from the outlet opening (112), thereby preventing any possible soiling of hands and clothes of the user.

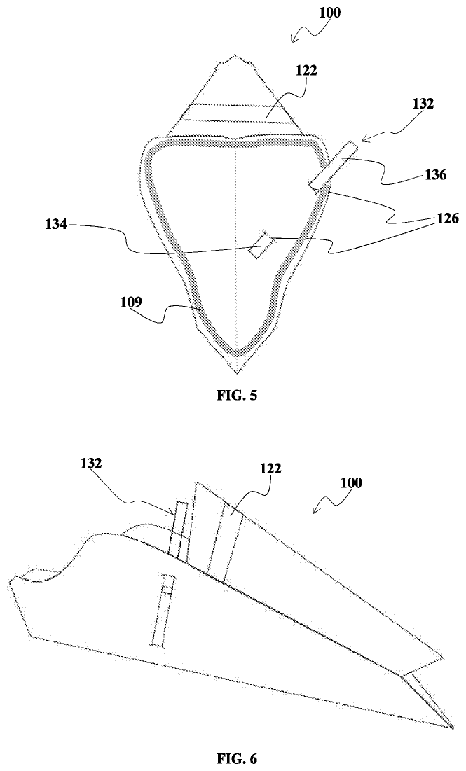

FIG. 5 illustrates a rear view of the urine testing device (100) shown in FIGS. 1 to 4, according to the exemplary embodiment of the present invention. The inlet opening (108) of the spout member (106) is adapted to be positioned to abut an exterior surface of the female urinary part to receive urine at the inlet opening (108). In the illustrated figure, the inlet opening (108) has a profile (125) adapted to conform to the body portion of the exterior surface of the female urinary part during urination and thereby creating an effective seal to prevent splashing or spillage of the urine during urination in a standing position or sitting position or squatting position. Further, the urine received through the inlet opening (108) will be discharged through the outlet opening (112) of the spout member (106).

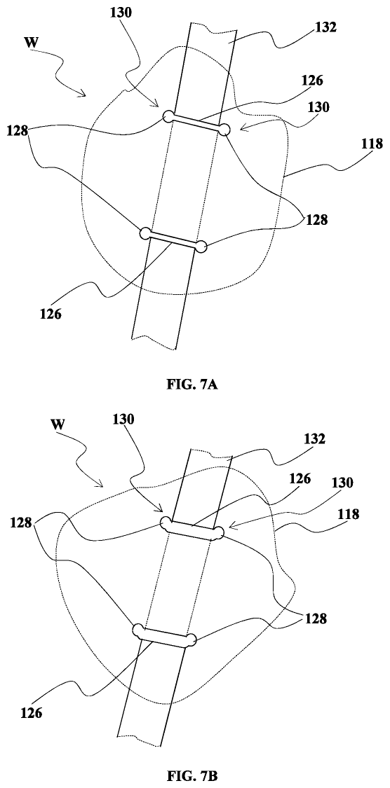

The urine testing device (100) further includes at least two slits (126) provided on any one of the wall members (118) of the spout member (106). It may be contemplated that, the wall member (118) may be provided with more than two slits (126). In the illustrated embodiment, the slits (126) are configured proximal to the inlet opening (108) of the foldable structure (102). In an embodiment, the slits (126) are provided with a hydrophobic layer for preventing spillage or leakage of urine through the slits (126). The slits (126) can be provided with relieving holes (128) on either ends (130) (shown in FIG. 7A and FIG. 7B) of each slit (126) for providing tear resistance during insertion of a test strip (132) into the slits (126) and to provide expanding feature for the slits (126) during insertion of the test strip (132). The slits (126) are provided on the wall member (118) by a machining process known in the art. Length of each slits (126) can be provided in such a way that, the slits (126) can accommodate and hold the test strip (132) firmly during packaging, transportation and while the usage of the device (100). Also, slit opening or size may be provided in a manner that will be sufficient to hold the test strip (132) without any external accessories like, glue or adhesive tapes. Thus, providing the advantage of the device (100) being cost effective. The slits (126) may be having a shape including, but not limited to, a straight line (shown in FIG. 7A), curved line like C-shape or U-shape forming a flap member (shown in FIG. 7B) in the wall member (118).

The test strip (132) of the device (100) is inserted into the slits (126) for testing the urine sample being allowed to flow into the device (100). In the illustrated embodiment, the test strip (132) is fixedly disposed in the slits (126) made in the wall members (118) of the spout member (106). The test strip (132) can be fixed to slits (126) the wall members (118) at an assembly stage at the factory. Thus, forming the device (100) being integrated with the test strip (132) and forming a ready kit for medical or diagnostic process. The test strip (132) is integrated at the manufacturing level. In the illustrated embodiment, the test strip (132) can be a pregnancy testing strip. The test strip (132) may however be any other test strip which can be used for testing other parameters or health conditions of the user. The device (100) as illustrated in the present disclosure may also be fixed with the test strip (132) apart from the pregnancy test strip. The device (100) may suitably be packaged accordingly.

The test strip (132) includes a testing portion (134) and an indicating portion (136). The testing portion (134) of the test strip (132) is disposed inside the spout member (106) and is adapted to come in contact with the urine flowing in the spout member (106) for testing the urine. The indicating portion (136) of the test strip (132) is disposed outside the spout member (106) for indicating the test results. Thus, the test results can be obtained by the user without removing the test strip (132) from the testing device (100), thereby providing a hygienic testing procedure for the female users. In other words, the test strip (132) allows capillary movement of the urine to a test area on the test strip (132) which provides visual indication of the presence or absence of an analyte in the urine on the outside of the wall members (118) of the device (100). Thus, the test strip (132) in the device (100) is adapted to optimally contact with the urine flowing in the testing device (100) and thereby to obtain more accurate results.

In the illustrated exemplary embodiment, the slits (126) in the wall member (118) is configured in such a way that the test strip (132) disposed in the slits (126) is positioned substantially vertical with respect to an axis (A-A′) (shown in FIG. 4) of the spout member (106) of the urine testing device (100) such that the testing portion (134) of the test strip (132) is substantially perpendicular to a flow path (not shown) of the urine. The phrases/terms “substantially perpendicular” or “substantially vertical” or “perpendicular” or “vertical” should be understood that, the position of the test strip (132) with respect to the wall member (118) or the spout member (106) may not be exactly vertical or perpendicular and that the position may be approximately vertical or perpendicular. The test strip (132) may or may not be exactly vertical or perpendicular to the axis (A-A′) of the spout member (106).

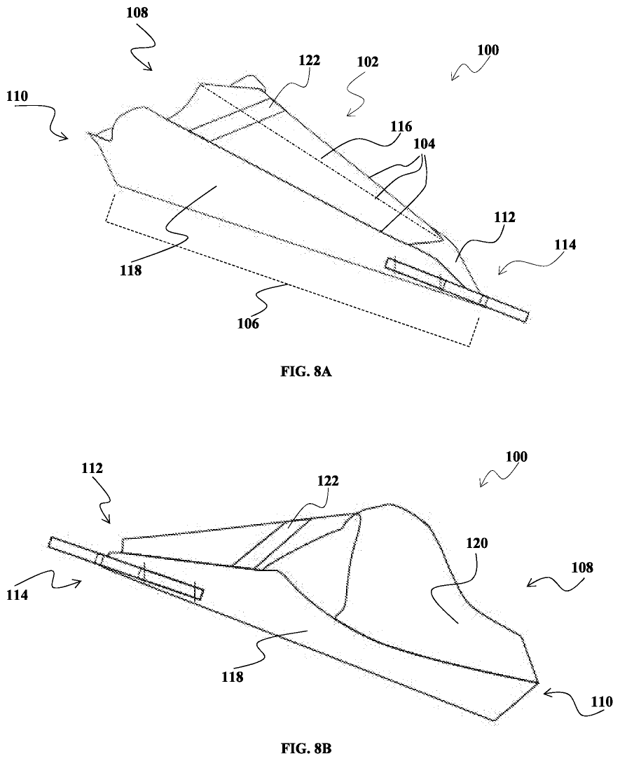

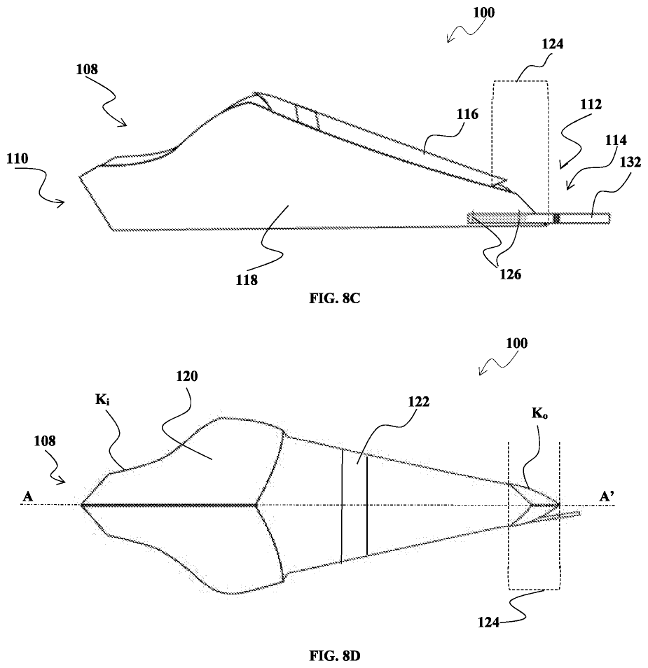

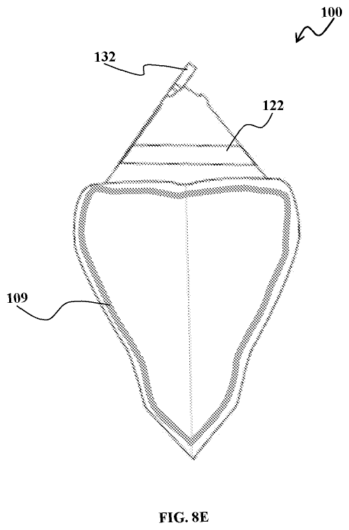

In another embodiment as shown in FIGS. 8A-8E, the slits (126) in the wall member (118) is configured in such a way that the test strip (132) disposed in the slits (126) is positioned substantially parallel with respect to the axis (A-A′) of the spout member (106) of the urine testing device (100) such that the test strip (132) is substantially parallel to flow path of the urine and the urine is allowed to flow over the testing portion (134) of the test strip (132). In an alternate embodiment, the test strip (132) may be affixed to the wall member (118) using glue or any other method known in the art to integrate the test strip (132) with the wall member (118). The phrase/term “substantially parallel” or “parallel” should be understood that, the position of the test strip (132) with respect to the wall member (118) or the spout member (106) may not be exactly parallel and the position may be approximately parallel. The test strip (132) may or may not be exactly parallel to the axis (A-A′) of the spout member (106).

Although the subject matter has been described in considerable detail with reference to exemplary embodiments thereof, other embodiments are possible. As such, the scope of the subject matter should not be limited to the description of the embodiments contained therein.

### Advantages of the Invention

In an embodiment, the device disclosed in the present disclosure is a simplified, hygienic, integrated, biodegradable, easy-to-carry, foldable, ready-to-use, simple to use, disposable or single-use yet economic device which can be used for home based self-testing of urine or can even be provided in public settings such as hospitals and diagnostic labs and do not require cumbersome and unhygienic method for collection of urine rather can be used even in standing or sitting position or squatting position in a hygienic manner. The device can be easily dispensed from a machine as well.

In an embodiment, the device disclosed in the present disclosure does not require assembling of any attachment during the testing procedure or any post procedure removal of the test strip from the device. Thus, the disclosed urine testing device provides an instant result without using other accessory and the cumbersome steps of inserting the test strip into the slots with due care and verifying the position of the test strip in the testing device by the user are also eliminated.

In an embodiment, the device disclosed in the present invention having the inside surface coated with the hydrophobic or water resistant material or a biodegradable polymer coating. Thereby the shape of the device is maintained during and after the urine test.

In an embodiment, the testing procedure using the device does not require any urine collection container or a cassette or even a dropper for conducting the urine test. Thus, the device disclosed in the present disclosure overcomes the limitations of prior arts and provides an easiest, cleanest, and a greener device which allows saving the planet earth from plastic pollution and lowering carbon footprint on the planet and eco-friendly urine testing process for various urine based tests.

In an embodiment, the device disclosed in the present disclosure having the spout member in tapered from the inlet opening to the outlet opening, and the longitudinal creases allows the device to be stored in a flattened manner or compact sized manner during non-working state of the device. Thus, the device can be easily carried along in a purse or vanity bags of the female users or even be used in dispensing machines in public places.

In an embodiment, the device disclosed in the present disclosure can be activated or deployed from a non-working condition to a working condition/state, by applying a small or little pressure on the longitudinal creases for pushing the longitudinal creases towards one another, thereby forming the wall members of the spout member to form the device. Thus, the device can be converted easily from the non-working condition to the working state.

The device disclosed in the present disclosure having the locking mechanism between the wall members allows the device to maintain the shape of the device like a stable structure in the open position/working condition. The open position of device forms a unique and ergonomic design at the inlet opening that fits easily abutting the exterior surface of the urethral area or the urinary part of the female user. Also, the unique curvature defined by the curved contour of the inlet opening/the inlet end, and the curvature in the back or lateral side are designed to prevent back-flow of the urine stream and does not provide any discomfort.

The inlet opening or receptacle of the device provided with the absorbent material and/or coating which come in close contact with the exterior surface of the urinary body part of the female users enhances the comfort since the absorbent material is a soft fabric like material. It also eliminates injuries to the contacting exterior surface of the female urinary part. The device is not required to be inserted inside the female urinary body part since the device forms a symmetrical curving of the inlet portion/opening to create an effective receptacle area when the device is in open position/configuration. Also, such receptacle area prevents splashing or spillage of fluids during urination in the standing position or the sitting position or the squatting position. That is to say, the profile of the inlet opening of the spout member is a reflection or symmetric curvature design that can easily abut the exterior surface of the urethral area or the urinary body part of the female user. This preferred unique curvature design allows the device to be safe whenever there is a contact with the body or skin in any positions like standing or sitting or squatting. The unique curvature captured by the curved contour of the inlet opening, rises higher towards a back or lateral side in order to prevent back-flow of the urine flow and also to collect even the last few drops of the urine so that clothes do not get soiled. Thus, hygiene is maintained during and after the process of urination and testing.

The device having the test strip which is fixed in the spout member at the slits at strategic points at least two levels, so the test strip remains non-movable during the testing procedure or when the device is folded. Also, the slits at strategic points ensures mid flow stream of urine to come in contact with the test strip over longer period and the entire test portion gets the flow. The testing strip is disposed and fixed in such manner that right test area comes in contact with urine and gets properly contacted with the urine flow to shown the result at the exterior portion of the device. Thus, the strip remains stable during the high pressure urine flow or stream to provide accurate result during testing procedure. Also, the strip is fixed and non-movable during storing and packaging. Also, since the device is packaged and sold in a flattened or straight form, the inlet opening and the wall members do not weaken and thus strength of the wall members and edges of the inlet opening of the device are maintained. Thereby the testing procedure can be carried out in a cost effective manner.

In an embodiment, the test strip can be placed vertically or at angular position, or horizontally, with respect to longitudinal direction or the axis of the device. Also, the test strip is disposed to allow only an optimum area of the strip to come in contact with urine. The test strip allows capillary movement of the urine to the test area on the strip which provides visual indication of the presence or absence of an analyte in the urine on the outside of the wall members of the device. Thus, the test strip in the device is adapted to optimally contact with the urine flowing in the testing device and thereby to obtain more accurate results.

In an embodiment, the slits or cuts provides a strategic and seamless integration of the testing strip into the wall members of the spout member in such a manner that the test results are visible on the outer side of wall member of the device for providing an indication for the presence or absence of analyte in the urine of the user.

In an embodiment, the user can purchase separate devices with integrated test strip for conducting tests for example, pregnancy, jaundice and urinary tract infection.

In an embodiment, the device disclosed in the present disclosure provides advantages over the existing device, by not adding any test strip to the device and thus reduces the chore to procure the device not having the test strip and separately adding the test strip to the device.

In an embodiment, the device disclosed in the present disclosure provides an advantage of easy operation, for example, press and use operation/mechanism, where the device just need to be brought from flattened or straight form to a workable form/deployed state for its use.

### INDUSTRIAL APPLICABILITY OF THE INVENTION

The disclosed urine testing device finds its potential application in the medical field such as, but not limited to, testing of urine by the female user. The urine testing may be made for diagnosis of medical condition such as pregnancy and various other diseases/health condition of a person. The testing device disclosed in the present disclosure may however find its applications in conducting other tests including, but not limited to, dope test to determine any possible consumption of drugs or steroids or any home based test including, but not limited to, jaundice, diabetes and urinary tract infection, for which urine works as an indicator.

While aspects of the present invention have been particularly shown and described with reference to the embodiments above, it will be understood by those skilled in the art that various additional embodiments may be contemplated by modification of the disclosed device without departing from the scope of what is disclosed. Such embodiments should be understood to fall within the scope of the present invention as determined based upon claims and any equivalents thereof.

### LIST OF REFERENCE CHARACTERS AND SIGNS EXPLAINED IN DETAILED DESCRIPTION AND DRAWINGS (FIGS. 1-6, 7A-7B AND 8A-8E)

100: Urine testing device or testing device or device
102: Foldable structure
104: Longitudinal creases
106: Spout member
108: Inlet opening
109: Absorbent material
110: Proximal end
112: Outlet opening
114: Distal end
116: Top member
118: Wall members
120: Inner surface
122: Locking mechanism
124: Elongated channel
125: Profile
126: Slits
128: Relieving holes
130: Ends of the slits
132: Test strip
134: Testing portion
136: Indicating portion
Kᵢ: Area of inlet opening
Kₒ: Area of outlet opening
A-A′: Axis of spout member

1-13. (canceled)

14. An integrated urine testing device for a female, the urine testing device comprising:
a foldable structure being formed from a sheet of material, defining a spout member having an inlet opening at a proximal end and an outlet opening at a distal end, the spout member comprising a top member and wall members, the inlet opening being adapted to be positioned abutting an exterior surface of a female urinary part to receive urine at the inlet opening, and to discharge the urine through the outlet opening;
at least two slits are configured on any one of the wall members of the spout member, the slits being configured proximal to the inlet opening of the foldable structure; and
at least one test strip comprising a testing portion and an indicating portion, the test strip being fixedly disposed at the slits of the wall member of the spout member;
wherein:
the testing portion of the test strip is disposed inside the spout member and is adapted to come in contact with the urine flowing in the spout member for testing the urine, and the indicating portion of the test strip is disposed outside the spout member for indicating the test results without the test strip being removed from the testing device;
the wall members of the foldable structure have at least one locking mechanism on its surface, and wherein the locking mechanism is adapted to prevent folding of the urine testing device during the usage, wherein the locking mechanism is a metallic strip which is inserted or affixed during manufacturing process right at top surface or outer surface of the device to ensure the grip and shape of the device stays firm; and
the slits are provided with a hydrophobic layer for preventing spillage of urine through the slits.

15. The integrated urine testing device of claim 14, wherein the inlet opening is provided with an absorbent material around its periphery for absorbing any possible spilled urine during urination.

16. The integrated urine testing device of claim 14, wherein the inlet opening has a profile adapted to conform to the exterior surface of a body portion of the female urinary part during urination and thereby creating an effective seal to prevent splashing or spillage of the urine during urination in a standing position or a sitting position or a squatting position.

17. The integrated urine testing device of claim 14, wherein the inlet opening has a larger size than the outlet opening forming a frustum of pyramid structure.

18. The integrated urine testing device of claim 17, wherein the inlet opening is provided with an absorbent material around its periphery for absorbing any possible spilled urine during urination.

19. The integrated urine testing device of claim 17, wherein the inlet opening has a profile adapted to conform to the exterior surface of a body portion of the female urinary part during urination and thereby creating an effective seal to prevent splashing or spillage of the urine during urination in a standing position or a sitting position or a squatting position.

20. The integrated urine testing device of claim 14, wherein:
the inlet opening is provided with an absorbent material around its periphery for absorbing any possible spilled urine during urination;
the inlet opening has a profile adapted to conform to the exterior surface of a body portion of the female urinary part during urination and thereby creating an effective seal to prevent splashing or spillage of the urine during urination in a standing position or a sitting position or a squatting position.

21. The integrated urine testing device of claim 20, wherein the inlet opening has a larger size than the outlet opening forming a frustum of pyramid structure.

22. The integrated urine testing device of claim 14, wherein the foldable structure has one or more longitudinal creases for folding the urine testing device before usage and for forming the wall members of the foldable structure of the testing device during the usage.

23. The integrated urine testing device of claim 14, wherein the slits in the wall member are configured in such a way that the test strip being disposed in the slits are positioned substantially vertical with respect to an axis of the spout member of the urine testing device such that the testing portion of the test strip is substantially perpendicular to flow path of the urine.

24. The integrated urine testing device of claim 14, wherein the slits are provided with relieving holes on either ends of the slits for providing tear resistance during insertion of the test strip into the slits.

25. The integrated urine testing device of claim 14, wherein the slits in the wall member are configured in such a way that the test strip being disposed in the slits are positioned substantially parallel with respect to the axis of the spout member of the urine testing device such that the test strip is substantially parallel to flow path of the urine and the urine is allowed to flow over the testing portion of the test strip.

26. The integrated urine testing device of claim 25, wherein the slits are provided with relieving holes on either ends of the slits for providing tear resistance during insertion of the test strip into the slits.

27. The integrated urine testing device of claim 14, wherein the material of the foldable structure is a biodegradable material.

28. The integrated urine testing device of claim 14, wherein the spout member has an inner surface coated with a hydrophobic material.

29. The integrated urine testing device of claim 14, wherein the outlet opening is provided with an elongated channel having a v-shaped cross section for guiding and increasing flow rate of the urine.
